(19) United States
(12) Patent Application Publication
(10) Pub. No.: US 2023/0015868 A1
(43) Pub. Date: Jan. 19, 2023

(54) Protective shield for odontological procedures

(71) Applicant: MIND MEDICAL INDUSTRIAL DESIGN, S. DE R.L. DE C.V., Chihuahua (MX)

(72) Inventors: Hugo FLORES TALAVERA, Chihuahua (MX); Martin Arturo CORRAL HERNANDEZ, Chihuahua (MX)

(73) Assignee: MIND MEDICAL INDUSTRIAL DESIGN, S. DE R.L. DE C.V., Chihuahua (MX)

(21) Appl. No.: 17/553,345

(22) Filed: Dec. 16, 2021

(30) Foreign Application Priority Data
Jul. 13, 2021 (MX) MX/U/2021/000372

(51) Int. Cl. A41D 13/11; A61C 19/00

(52) U.S. Cl. CPC A41D 13/1138; A61C 19/007; A62B 23/025

(57) ABSTRACT

The containment of aerosols produced in dental processes and treatments is of notorious relevance since they represent a high risk of transmission of infectious diseases, such as viral flu or COVID-19. Medical personnel in the dental area, or others, are exposed to a high risk of infection by constant exposure to these aerosols and splashes. Consequently, it is providing a protective shield for odontological procedures that functions as a physical barrier avoiding contact of dentists, orthodontists and/or their technicians/assistants with aerosols and splashes that occur during dental treatments and procedures.

### BACKGROUND OF THE INVENTION

### Object of the Invention

The object of the present invention is to provide a protective shield for odontological procedures that functions as a physical barrier avoiding contact of dentists, orthodontists and/or their technicians/assistants with the aerosols and splashes that occur during dental treatments and procedures.

### FIELD OF THE INVENTION

The present invention belongs to the technical field of specially adapted accessories for the medical sciences.

### DESCRIPTION OF THE RELATED ART

During the performance of oral procedures, dentists, orthodontists and/or technicians/assistants may be in constant contact with the aerosols emanating from a patient, which represents a risk to their health. For this reason, the containment of aerosols produced in dental processes and treatments is of notorious relevance since they represent a high risk of transmission of infectious diseases, such as viral flu or COVID-19. The medical personnel of areas such as dental, or other similar, are exposed to a high risk of infection by the constant exposure to these aerosols and splashes, which results in the necessity to generate solutions that minimize contact with these aerosols while allowing the best practices to be carried out.

Within the previous state of the art, in the matter of inventions, regarding protection against splashes in oral procedures, one can mention the document number CN106535814A, which refers to a protective cover for a field of surgical operations. Such protective cover comprises a partition that separates a protection zone from an outer chamber, and a protective support surface that surrounds the operational field. According to the invention, the partition comprises a through opening which is surrounded by a sealing surface for a shaft of a surgical instrument. The protective cover is intended to be placed on the surface surrounding the surgical field by means of a support surface. The protective cover is defined on one side by the field of operation or the surrounding area of the field of operation, and by the partition on the other side. If the protective cover is in a separate state, the protective chamber passes into the environment through the opening surrounded by the support surface. The support surface is designed to surround the surgical field.

On the other hand, document number CN111202591A refers to an insulator for the treatment of the oral cavity, which includes an insulation cover, an adapter for devices and a laminator. The insulator is fixed above a patient's oral cavity; the insulation cover is transparent and therefore a good field of view is provided for treatment operations; the device adapter is an elastic component that provides an opening access for a treatment device and forms a seal by adapting to an outer diameter of the appliance; the insulation cover is fixed firmly on the patient's oral cavity by the laminator, and a seal is formed by adapting to the surface of an object wrapped by the insulation cover, in such a way that gases and liquids generated by the treatment are contained in the insulator. The surface of the insulation cover is provided with a discharge opening that is connected to a discharge pipe, so the gases and liquids are leaded to a waste treatment system in a directed way through the discharge pipe, so that cross-infection is effectively prevented, while treatment safety and environment are improved.

Similarly, document number CN111760156A describes a jet-proof aerosol insulating cover comprising an inner coverage and an outer coverage; where the inner cover has a cubical structure with an unclosed bottom; the outer coverage has a cubical structure with both top and bottom open; the inner cover is embedded in the outer coverage from top to bottom in a slide-out manner; the upper face of the inner coverage has a stepped window structure; the stepped window structure consists of an upper layer baffle and a lower layer baffle; the left side and the right side of the lower layer baffle are each provided with a pressure groove; the upper layer baffle is in a nested connection over the bottom layer baffle through the left and right pressure grooves, in a slide-out manner; it also includes a window for the mouth and nose in a middle area of the lower layer baffle; the interior coverage is provided with a threaded tube window; a neck window is kept in an intermediate position, near the bottom, on the back face of the outer covering; and the outer covering is provided with glove box-type windows. It does not cause hypoxia or asphyxia in patients, and tracheal intubation is not affected by the use of the isolation cover.

Additionally, document number CN111374786A describes a negative pressure oral grip capable of preventing the diffusion of aerosols, comprising a gag body and a transparent box that covers the mouth. Where the body of the gag comprises supports for the lips, used to open the lips, and a saliva suction cover attached to the wall of an oral cavity; the suction holes are distributed on the inner surfaces of the box covering the mouth and the saliva suction cover, and communicate with a first negative pressure generating unit; the box covering the mouth comprises a part covering the mouth connected to the peripheries of the lips in a sealed mode, and an operating box fixed to the part covering the mouth; the support columns are connected and arranged in the operation box in a pivoting mode; a flexible operating opening is arranged in the operating box in a sealed mode; a magnifying lens is embedded on one side of the flexible operating opening; and a palm rest rack is arranged in the operating box.

There is also document number CN111281571A, which refers to a protective insulation cover for oral surgery operations, comprising an insulating cover body formed by supporting a mask layer by a supporting frame, and provided with an accommodation space; in which the body of the insulation cover comprises a side wall of the mask, a front part of the mask, a back part of the mask and an opening part; the side wall of the mask comprises a left side wall, a right side wall and a lower side wall; the front of the mask is transparent; the operating entrances are formed on the left sides of the left side wall and the lower side wall; a first negative pressure suction port is formed on the right side wall; the opening part is arranged on the back or on the lower side wall of the mask; during use, a patient's head is located in the accommodation space and an operating space is formed between the face and the front of the mask. The isolation cover is particularly suitable for diagnosis and oral treatment in the period of prevention of infectious diseases.

Likewise, document number CN111685953A describes a multifunctional isolation device comprising a transparent cover capable of covering the head of a human body, in which the transparent cover is shaped like a quadrangular trunk pyramid, and the size of the face of the upper bottom of the transparent cover is smaller than that of the face of the lower bottom of the transparent cover; On the face of the upper bottom of the transparent cover, two operating holes are formed that allow the penetration of the hands, and on the other side of the transparent cover, opposite the operating holes, an opening is formed that allows a patient's head to be placed inwards; a disinfection assembly is arranged in each operating hole, in which each disinfection assembly comprises fixing ears, strip-shaped turntables, a sliding fixation rod, a wet disinfectant part and a fastening buckle. Hand isolation and disinfection protection can be provided to medical personnel when performing intubation to patients with respiratory failure, reducing infection of medical personnel and cross-infection between patients.

In addition, document number CN111281321A refers to a multifunctional protective mask for oral diagnosis and treatment, comprising an upper nasal protection area, a nasal surface protection area, an eye protection area, a gag, a submandibular protection area and a fixation band. The nasal surface protection area is used to cover and protect the patient's nasal surface; the eye protection area is a visual window for communication between doctors and patients in the treatment process; the soft plastic elastic gag is arranged in the protective area of the nose and face, and can support the open lip area to protect the soft tissue after being placed in the patient's oral cavity, facilitating the suction of saliva; the submandibular protection area is arranged below the nasal and facial protection area, and is used to cover the soft tissues of the submandibular area; the fixation band is arranged in the nasal surface protection area and is used to attach the nasal surface protection area to a user's face; the mask is able to help open a patient's lip tissue and keep it stable, preventing cross-infection between patients.

Finally, document number CN111631834A describes a protective device comprising a protective cover, which in turn comprises a visual window and a cover body used to cover a patient. Where the visual window is connected to the cover body in a sealed mode, the cover body is provided with an opening and an operating hole, the opening is used to allow a medical instrument to extend into the cover body, and the operating hole is used to allow a doctor's arm to extend into the cover body; one end of a suction pipe communicates with the body of the cover, and the other end is used to connect to an external suction device. The cover body can block the diffusion of aerosols or splashes, as these are collected in it and then sucked by the external suction device through the suction tube, so that the aerosol is sucked once generated, and no aerosol remains in the corresponding space in the cover body after removing it from the patient.

Once the above is exposed, it is evident that there are currently a variety of protective covers for oral procedures that somehow resemble the shield object of the present invention, however, among the main technical advantages of this invention are its disposable nature and its folding and compact structure, which allow it to be packaged, stored and transported efficiently. In addition, the present invention has the ability to self-assemble, since when opening the container for use, the steel tensioner is automatically deployed and creates the desired shape, without the need to assemble piece by piece, thus avoiding technical complications. It should be noted that the shield is made mostly of breathable material, which allows the exchange of gases such as oxygen and carbon dioxide between the outer and inner space of the capsule.

### BRIEF DESCRIPTION OF THE DRAWINGS

FIG. 1. Front view of the protective shield for odontological procedures.

FIG. 2. Top view of the protective shield for odontological procedures.

FIG. 3. Bottom view of the protective shield for odontological procedures.

FIG. 4. Right side view of the protective shield for odontological procedures.

FIG. 5. Left side view of the protective shield for odontological procedures.

FIG. 6. View of the protective shield placed over the head of a user or patient.

FIG. 7. View of the folded protective shield.

### DETAILED DESCRIPTION OF THE INVENTION

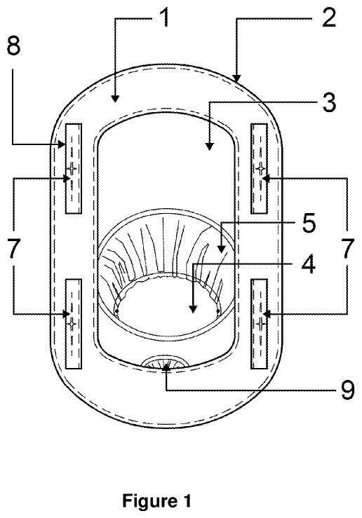

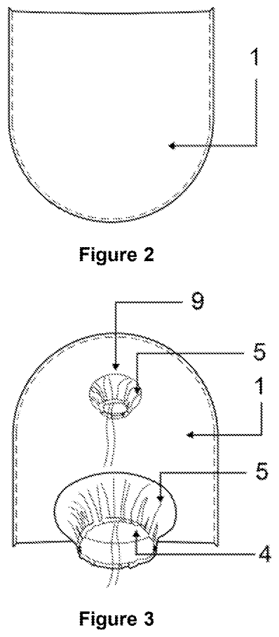

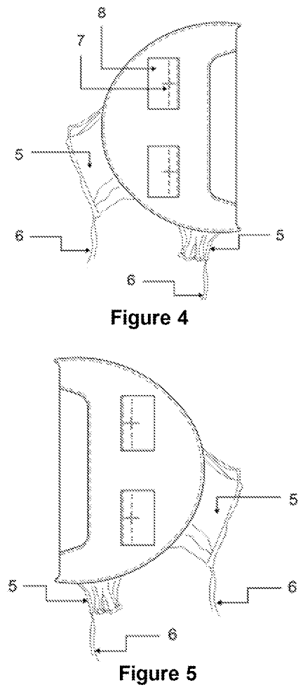

The present invention describes a protective shield for odontological procedures that functions as a physical barrier avoiding contact of dentists, orthodontists and/or their technicians/assistants with aerosols and splashes that occur during dental treatments and procedures. It is comprised of an empty capsule (1) that covers the head of a user to contain the aerosols inside it, reducing the spread of aerosols to the outside, where said capsule (1) has a self-folding flexible frame (2) that allows to extend the capsule (1) to place it on the user's head, or fold the capsule (1) to reduce its extension and store it, transport it or discard it. The capsule (1) has a transparent screen (3) in the center so that it enables the user to observe to the outside, and who is performing a procedure on the user to observe towards the inside of the capsule (1); at the bottom of the protective shield is an inlet hole (4) through which the user's head is inserted, said inlet hole (4) has a sleeve (5) that allows to adjust the width of the inlet hole (4) and seal the capsule (1) in the neck of the user, where the adjustment is made by adjustable laces or strips (6). On the side of the protective shield are working holes (7) that function as access, for the hands of the person who is performing a procedure on the user, to place inside the capsule (1) instruments and materials required for the procedure; these working holes (7) have a cover (8) that serves as a mechanism to cover or close them as soon as they are not required, finally the protective shield has a nozzle (9) that serves to adapt various extraction instruments that are required for a long time, where the nozzle (9) has a sleeve (5) that allows to adjust the width of it through the use of adjustable laces or strips (6). FIGS. 1, 2, 3, 4 and 5 show the front, top, bottom, right side and left side view of the components that make up the protective shield, respectively.

### Preferred Embodiment of the Invention

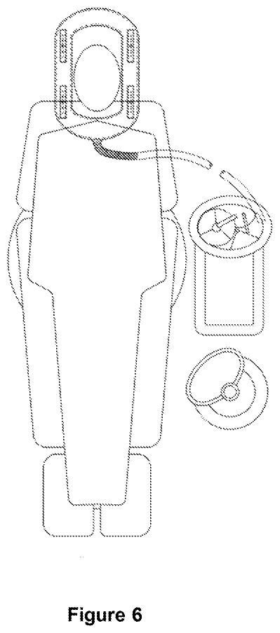

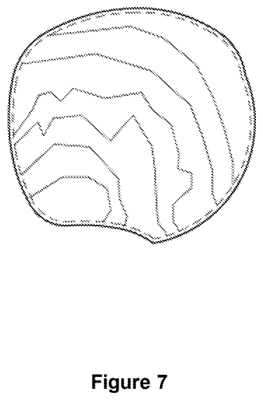

The protective shield for odontological procedures is placed on the head of a user or patient (as shown in FIG. 6) and consists of an empty capsule (1) that covers the head of a user to contain the aerosols inside it, forming a physical barrier that reduces the spread of aerosols of the patient to the outside of the capsule (1), and the spreading of infectious material during medical or dental procedures in the head or oral cavity of the patient type user, where said capsule (1) has a self-folding flexible frame (2) that allows to extend the capsule (1) to place it on the user's head or fold the capsule (1) to reduce its extension and store it, transport it or discard it; said flexible frame (2) is preferably a steel wire core of rectangular section, joined at its ends forming an oval, which allows the protective shield to be folded to a small, flat and circular shape (as shown in FIG. 7) to be packed, stored and transported efficiently. When opening the packaging of the protective shield the flexible frame (2) opens and creates the desired shape without the need to assemble, the flexible frame can be made of steel wire of various similar spring constants. The capsule (1) is mostly made of breathable material that allows the exchange of gases such as oxygen and carbon dioxide between the inside and outside space of the protective shield, while restricting the transport of liquids such as aerosols. Some breathable materials include (to mention examples, without limiting the invention) non-woven fabric, such as “spunbond” polypropylene.

The capsule (1) has a transparent screen (3) in the center so that it enables the user to observe outwards, and whoever is performing a procedure on the user to observe towards the inside of the capsule (1); the transparent screen (3) is located in the middle of the capsule (1) in a way that enables the user who performs a procedure on the user of the protective shield (doctor and medical staff) to see the face of the user of the protective shield (patient) and the procedure performed on the patient, without restriction. It also enables the patient to have quasi-normal vision of the environment. The transparent screen (3) prevents fogging and can be made, for example, of polyvinyl chloride. In the lower part of the protective shield is an inlet hole (4) through which the user's head is inserted, where said inlet hole (4) has a sleeve (5) that allows to adjust the width of the inlet hole (4) and seal the capsule (1) in the user's neck, where the adjustment will be made by adjustable laces or strips (6). On the side of the protective shield are working holes (7) that function as access for the hands of the person who is performing a procedure on the user, for the placement of instruments and materials that are required for the procedure inside the capsule (1), these working holes (7) have a cover (8) that serves as a mechanism to cover or close them as soon as they are not required, the protective shield has preferably four working holes (7) distributed as two holes on each side of the capsule (1), however, in other embodiments all those that are necessary can be included. Finally, the protective shield has a nozzle (9) that serves to adapt various extraction instruments that are required for a long time (for example: a vacuum cleaner, inhaler or another instrument), the nozzle (9) has a sleeve (5) that allows to adjust the width of it by using adjustable laces or strips (6).

The protective shield is manufactured using various manufacturing technologies that include (but are not restricted to) sewing, different welds, gluing processes and may include adjustable zippers, strips and/or laces. The device is intended to be disposed after use by each patient.

Having sufficiently described my invention, I consider as a novelty and therefore claim as my exclusive property, what is contained in the following clauses:

1. A protective shield for odontological procedures comprising:
an empty capsule (1) adapted to cover a head of a user to contain aerosols inside it and reducing the spread of aerosols to the outside, where said capsule (1) includes: a self-folding flexible frame (2) that allows to extend the capsule (1) to place on the head of the user or to fold the capsule (1), a transparent screen (3) in a center of the capsule to enable the user to observe the outside and to an user performing a procedure on the user to observe towards the inside of the capsule (1);
an inlet hole (4) located at the bottom of the protective shield where the user's head is inserted,
said inlet hole (4) has a sleeve (5) that allows to adjust the width of the inlet hole (4) and seal the capsule (1) in a neck of the user, wherein the adjustment is made by adjustable laces or strips (6),
working holes (7) located on a side of the protective shield that function as access for the hands of the user performing a procedure for the placement of instruments and materials that are required for the procedure inside the capsule (1), wherein the working holes (7) have a cover (8) that serves as a mechanism to cover or close the working holes as soon as they are not required,
a nozzle (9) to adapt various extraction instruments that are required for a long time, the nozzle (9) has a sleeve (5) that allows to adjust the width through the use of adjustable laces or strips (6).

2. The protective shield according to claim 1, wherein the flexible frame (2) includes a flat section steel wire attached at its ends, that forms an oval, which allows the protective shield to be folded to a small, flat and circular shape to be packed, stored and transported efficiently.

3. The protective shield according to claim 1, wherein the capsule (1) is made of breathable material that allows the exchange of gases between the space outside and inside the protective shield, while restricting the transport of liquids as aerosols.

4. The protective shield according to claim 1, wherein the transparent screen (3) prevents fogging and is made of polyvinyl chloride.

5. The protective shield according to claim 1, wherein the protective cover has four working holes (7) distributed as two holes on each side of the capsule (1).
